(19) United States
(12) Patent Application Publication
(10) Pub. No.: US 2021/0068960 A1
(43) Pub. Date: Mar. 11, 2021

(54) Protective Sheath For Medical Implant

(71) Applicant: Spinal Balance, Inc., Swanton, OH (US)

(72) Inventors: Faezah Agarwal, Swanton, OH (US); Aakash Agarwal, Swanton, OH (US)

(73) Assignee: Spinal Balance, Inc., Swanton, OH (US)

(21) Appl. No.: 16/958,837

(22) PCT Filed: Dec. 28, 2018
(86) PCT No.: PCT/US18/67883

§ 371 Date: Jun. 29, 2020

Related U.S. Application Data
(60) Provisional application No. 62/611,948, filed on Dec. 29, 2017.

(51) Int. Cl. A61F 2/30; A61F 2/36; A61F 2/34; A61F 2/38

(52) U.S. Cl. CPC A61F 2/30734; A61F 2/3609; A61F 2/34; A61F 2/3859; A61F 2002/3435; A61F 2002/30738; A61F 2002/3625; A61F 2002/3611; A61F 2002/30718

(57) ABSTRACT

A combined assembly of a medical implant and a sheath includes a medical implant having a surface and a sheath that is releasably secured to and protectively covers the surface of the medical implant. The sheath may protectively cover some or all of the surface of the medical implant. The sheath may be frictionally or mechanically retained on the medical implant. The medical implant may include both an inner surface and an outer surface, and the sheath may include both an inner sheath portion and an outer sheath portion. The sheath may include one or more perforations, a gripping structure that cooperates with a handling tool, or a handle.

### CROSS-REFERENCE TO RELATED APPLICATIONS

This application claims the benefit of United States Provisional Application No. 62/611,948, filed Dec. 29, 2017, the disclosure of which is incorporated herein by reference.

### BACKGROUND OF THE INVENTION

This invention relates in general to medical implants. In particular, this invention relates to a removable sheath that temporarily covers a portion of a surface of such a medical implant to protect the surface from exposure to contaminants prior to installation in a human or animal body during a surgical procedure.

Medical implants are artificial prosthetic devices that are commonly used to replace damaged body parts, such as joints and limbs, and for other reasons. A medical implant may be formed from either a single component or a plurality of components that function together as an assembly. A wide variety of medical implants are known in art and are commonly used for many medical applications.

The installation of a medical implant in the body can be difficult because of the requirements for properly positioning and orienting the implant. In some instances, the installation of the medical implant includes preliminarily using one or more of a variety of temporary trial components (hereinafter trials) that can be used to determine the proper size, fit, and relative orientation of the permanent implants. Subsequently, each trial is replaced by the permanent implant. Thus, installing an implant involves retrieving the trials and/or implants from a container, handling the trials and/or implants, delivering the trials and/or implants to the body, positioning and repositioning the trials and/or implants within the body, and removing the trials from the body.

The known process of retrieving, handling, delivering, positioning and repositioning, and removing trials and implants exposes any uncovered surfaces of the trials and implants to contamination by contact with gloves worn by the surgeon or surgical assistant, the wound of the patient, ambient air in an operating room, other surgical instruments, and other surfaces in the vicinity of the surgical environment prior to the final delivery of the trial or implant into the designated space in the body of the patient. Bacterial contamination can lead to infection and other adverse host tissue responses in the patient. Therefore, it would be advantageous to provide a device or method that reduces or eliminates the exposure of the surfaces of the trial or implant to contamination.

### SUMMARY OF THE INVENTION

This invention relates to a removable sheath that temporarily covers a portion of a surface of a medical trial or implant to protect the surface from exposure to contaminants prior to installation in a human or animal body during a surgical procedure. The medical implant has a surface, and the sheath is releasably secured to and protectively covers the surface of the medical implant. The sheath may protectively cover some or all of the surface of the medical implant. The sheath may be frictionally or mechanically retained on the medical implant. The medical implant may include both an inner surface and an outer surface, and the sheath may include both an inner sheath portion and an outer sheath portion. The sheath may include one or more perforations, a gripping structure that cooperates with a handling tool, or a handle.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of a conventional medical implant that includes a plurality of components.

FIG. 2 is an exploded perspective view of the plurality of components of the conventional medical implant illustrated in FIG. 1.

FIG. 3 is a perspective view of a container containing a first embodiment of a protective sheath in accordance with this invention that is attached to a first one of the medical implant components illustrated in FIG. 2.

FIG. 4 is a perspective view of the first embodiment of the sheath and the first medical implant component illustrated in FIG. 3 after removal from the container.

FIG. 5 is an exploded perspective view showing an outer sheath portion of the first embodiment of the sheath after being separated from the first medical implant component and an inner sheath portion of the first embodiment of the sheath illustrated in FIG. 4.

FIG. 6 is a cross sectional view of the first medical implant component and the inner sheath portion of the first embodiment of the sheath shown in FIG. 5.

FIG. 7 is an exploded perspective view of the inner sheath portion of the first embodiment of the sheath shown separated from the first medical implant component illustrated in FIG. 6.

FIG. 8 is a perspective view of a second embodiment of an inner sheath portion in accordance with this invention.

FIG. 9 is a perspective view of a third embodiment of an inner sheath portion in accordance with this invention.

FIG. 10 is a perspective view of a fourth embodiment of an inner sheath portion in accordance with this invention.

FIG. 11 is a perspective view of the fourth embodiment of the inner sheath portion together with an installation/removal tool.

FIG. 12 is a perspective view of a fifth embodiment of a sheath in accordance with this invention.

FIG. 13 is a perspective view of a sixth embodiment of a sheath in accordance with this invention.

FIG. 14 is a perspective view of a seventh embodiment of a sheath in accordance with this invention that is attached to a second one of the medical implant components illustrated in FIG. 2.

FIG. 15 is a cross sectional view of the second medical implant and the seventh embodiment of the sheath illustrated in FIG. 14.

FIG. 16 is an exploded perspective view of the second medical implant and the seventh embodiment of the sheath illustrated in FIGS. 14 and 15.

FIG. 17 is a perspective view of an eighth embodiment of a sheath in accordance with this invention that is attached to the second one of the medical implant components illustrated in FIG. 2.

FIG. 18 is a side elevational view of a ninth embodiment of a sheath in accordance with this invention that is attached to a third one of the medical implant components illustrated in FIG. 2.

FIG. 19 is a cross sectional view of the ninth embodiment of the sheath and the third medical implant component illustrated in FIG. 18.

FIG. 20 is an exploded side elevational view of the ninth embodiment of the sheath and the third medical implant component illustrated in FIGS. 18 and 19.

FIG. 21 is a side elevational view of a tenth embodiment of a sheath in accordance with this invention that is attached to the third one of the medical implant components illustrated in FIG. 2.

FIG. 22 is an exploded side elevational view of the tenth embodiment of the sheath and the third medical implant component illustrated in FIG. 21.

FIG. 23 is a perspective view of an eleventh embodiment of a sheath in accordance with this invention.

FIG. 24 is a perspective view of another conventional medical implant that includes a plurality of components.

FIG. 25 is an exploded perspective view of the plurality of components of the conventional medical implant illustrated in FIG. 24.

FIG. 26 is a front perspective view of a twelfth embodiment of a sheath in accordance with this invention that is attached to a first one of the components of the conventional medical implant illustrated in FIGS. 24 and 25.

FIG. 27 is a rear perspective view of the twelfth embodiment of the sheath and the first one of the components of the conventional medical implant illustrated in FIGS. 24 and 25.

FIG. 28 is an exploded front perspective view of the twelfth embodiment of the sheath and the first one of the components of the conventional medical implant illustrated in FIGS. 24 and 25.

FIG. 29 is an exploded rear perspective view of the twelfth embodiment of the sheath and the first one of the components of the conventional medical implant illustrated in FIGS. 24 and 25.

FIG. 30 is a perspective view of a thirteenth embodiment of a sheath in accordance with this invention that is attached to a second one of the components of the conventional medical implant illustrated in FIGS. 24 and 25.

FIG. 31 is an exploded perspective view of the thirteenth embodiment of the sheath and the second one of the components of the conventional medical implant illustrated in FIG. 30.

FIG. 32 is cross sectional view of the thirteenth embodiment of the sheath and the second one of the components of the conventional medical implant illustrated in FIGS. 30 and 31.

FIG. 33 is a perspective view of a fourteenth embodiment of a sheath in accordance with this invention that is attached to a third one of the components of the conventional medical implant illustrated in FIGS. 24 and 25.

FIG. 34 is an exploded perspective view of the fourteenth embodiment of the sheath and the third one of the components of the conventional medical implant illustrated in FIG. 33.

### DETAILED DESCRIPTION OF THE PREFERRED EMBODIMENTS

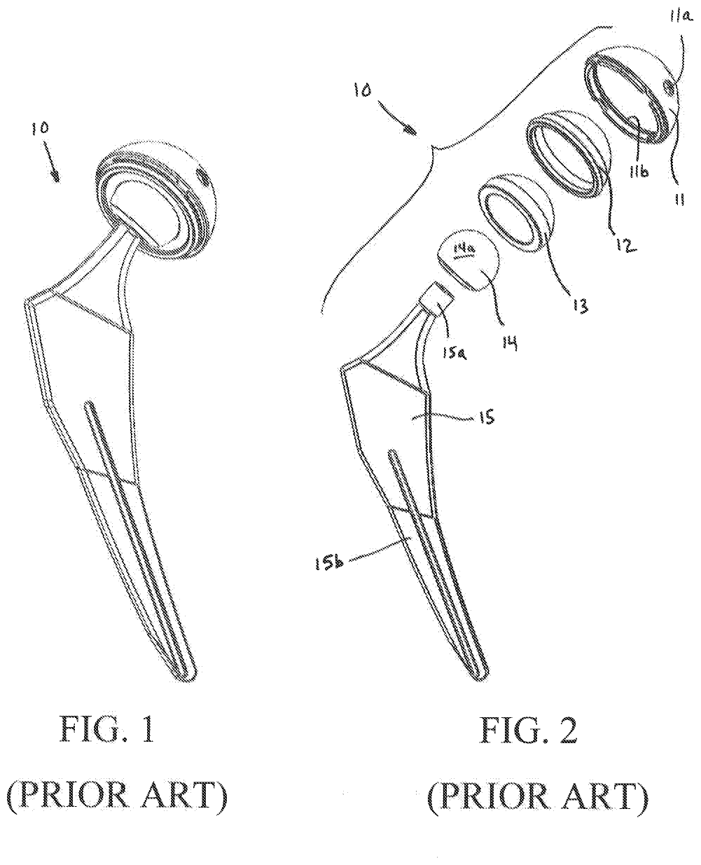

Referring now to the drawings, there is illustrated in FIGS. 1 and 2 a medical implant, indicated generally at 10, that is conventional in the art. The illustrated conventional medical implant 10 is a hip implant assembly that includes an acetabular shell 11, a liner 12, a bearing 13, a femoral head 14, and a femoral stem 15. The acetabular shell 11 of the hip implant assembly 10 is intended to be secured to a hip of a patient, while the femoral stem 15 of the hip implant assembly 10 is intended to be secured to a femur of the patient. The liner 12, the bearing 13, and the femoral head 14 are connected between the acetabular shell 11 and the femoral stem 15 so as to facilitate relative rotational movement therebetween. It should be understood, however, that the illustrated conventional hip implant assembly 10 is intended to be representative of any individual device or combination of devices intended for installation in a human or animal body during a surgical procedure. Thus, the scope of this invention is not intended to be limited to the specific structure of the illustrated conventional hip implant assembly 10. On the contrary, as will become apparent below, this invention may be used in any desired environment and for any desired purpose.

The illustrated acetabular shell 11 is shaped generally in the form of a hollow semi-sphere having an outer surface and an inner surface. A plurality of apertures Ila extends through the acetabular shell 11 from the outer surface to the inner surface. The apertures 11a are adapted to receive respective threaded fasteners (not shown) that can secure the acetabular shell 11 to the hip of the patient. The inner surface of the acetabular shell 11 has an annular groove 11b formed therein for a purpose that will be explained below.

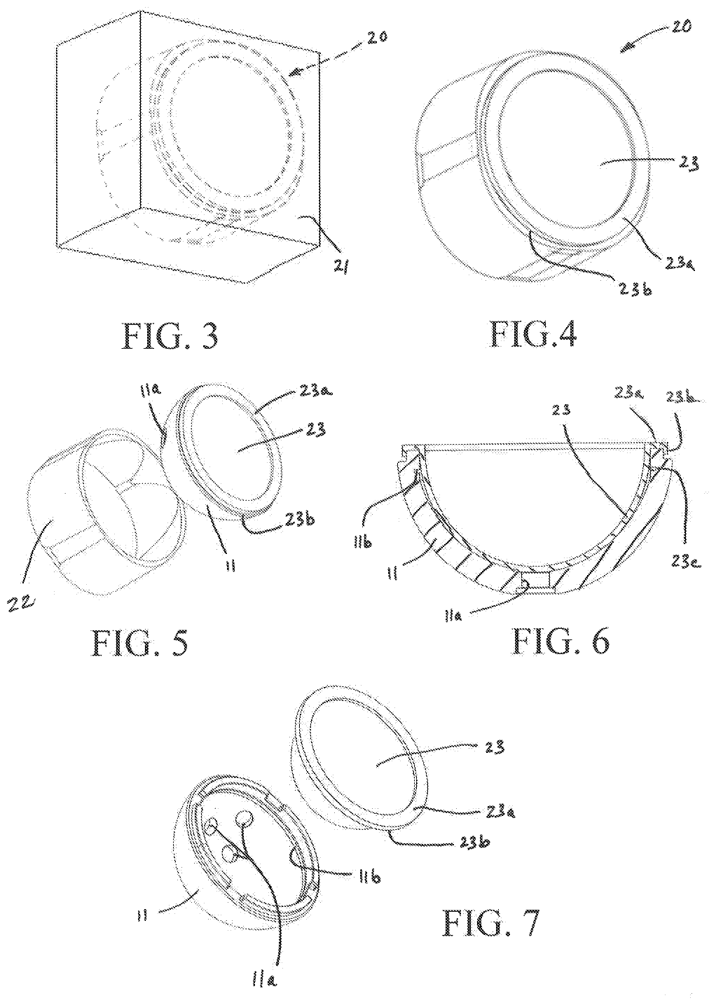

FIGS. 3 through 7 illustrate a first embodiment of a protective sheath, indicated generally at 20, in accordance with this invention. As shown therein, the first embodiment of the sheath 20 is provided in connection with the acetabular shell 11, although such is not required. The combined assembly of the acetabular shell 11 and the first embodiment of the sheath 20 is preferably pre-assembled in the configuration shown in FIG. 4, sterilized as a unit, and disposed within a protective container 21 as shown in FIG. 3 for delivery to the site of the operation for implantation. Such pre-assembly and sterilization facilitate the installation of the acetabular shell 11 in the patient as described below, with little or no manual handling of the acetabular shell 11 by the surgeon or assistant. As is well known, it is very important to minimize or eliminate such manual handling of the acetabular shell 11 (or any other medical implant, for that matter) to reduce the likelihood of undesirable contamination.

FIGS. 4 through 7 illustrate the structure of the first embodiment of the sheath 20 in greater detail. As shown therein, the first embodiment of the sheath 20 includes both an outer sheath portion 22 and an inner sheath portion 23. The outer sheath portion 22 is generally hollow and cylindrical in shape, although such is not required. The outer sheath portion 22 is preferably sized and shaped to conform with and frictionally engage the outer surface of the acetabular shell 11. However, the outer sheath portion 22 may engage and be retained on the acetabular shell 11 in any desired manner. When installed on the acetabular shell 11 as shown in FIG. 4, the outer sheath portion 22 functions as a physical barrier that protects the outer surface of the acetabular shell 11 from direct exposure to various sources of contamination, such as from the gloves of a surgeon or an assistant, wound edges, and other sources of contamination.

In the illustrated embodiment, an inner surface of the outer sheath portion 22 conforms with and covers the entire outer surface of the acetabular shell 11. However, if desired, the inner surface of the outer sheath portion 22 may only partially cover a portion of the outer surface of the acetabular shell 11. The inner surface of the outer sheath portion 22 may have any desired size and shape suitable to accomplish this. An outer surface of the outer sheath portion 22 is preferably sized and shaped to conform with an interior space defined by the protective container 21. This prevents relative movement from occurring between the first embodiment of the sheath 20 and the protective container 21 during shipment. However, the outer surface of the outer sheath portion 22 may have any desired size or shape.

The structure of the inner sheath portion 23 of the first embodiment of the sheath 20 is best illustrated in FIGS. 6 and 7. As shown therein, the inner sheath portion 23 is generally hollow and semi-spherical in shape, although again such is not required. In the illustrated embodiment, an outer surface of the inner sheath portion 23 conforms in shape with and covers the entire inner surface of the acetabular shell 11. However, if desired, the outer surface of the inner sheath portion 23 may only partially cover the inner surface of the acetabular shell 11. The illustrated inner sheath portion 23 has a generally circular rim that, as best shown in FIG. 6, covers a corresponding circular end portion of the acetabular shell 11. The illustrated rim of the inner sheath portion 23 is generally L-shaped in cross section, including a radially extending leg portion 23a that extends laterally across the end portion of the acetabular shell 11 and an axially extending leg portion 23b that extends across a portion of the outer surface of acetabular shell 11. Additionally, as best shown in FIG. 6, the illustrated inner sheath portion 23 also includes an annular ridge 23c that extends about the outer surface thereof. The annular ridge 23c is sized and shaped to extend within the annular groove l lb formed in the acetabular shell 11 to releasably and mechanically retain the inner sheath 23 thereon. Both the L-shaped rim and the annular ridge 23c are optional.

The manner in which the acetabular shell 11 is removed from the protective container 21 and provided for use at a surgical site will now be described. Initially, as discussed above, the combined assembly of the acetabular shell 11 and the first embodiment of the sheath 20 is preferably pre-assembled in the configuration shown in FIG. 4, sterilized as a unit, and disposed in the protective container 21 as shown in FIG. 3 for delivery to the site of the operation for use. Next, as shown in FIG. 4, the combined assembly of the acetabular shell 11 and the first embodiment of the sheath 20 is removed from the protective container 21. Because the first embodiment of the sheath 20 includes both the outer sheath portion 22 (which protectively covers the outer surface of the acetabular shell 11) and the inner sheath portion 23 (which protectively covers the inner surface of the acetabular shell 11), the removal of the combined assembly from the protective container 21 can be accomplished quickly and easily, and most importantly without touching or otherwise potentially contaminating the acetabular shell 11.

Next, as shown in FIG. 5, the outer sheath portion 22 is removed from the assembly of the acetabular shell 11 and the inner sheath portion 23. Because the L-shaped rim of the inner sheath portion 23 extends across both the end portion and a portion of the outer surface of the acetabular shell 11, this removal of the outer sheath portion 22 can also be accomplished quickly and easily, without touching or otherwise potentially contaminating the acetabular shell 11. Following such removal, the surgeon or assistant may position the assembly of the acetabular shell 11 and the inner sheath portion 23 at a desired location by manually grasping the L-shaped rim of the inner sheath portion 23, without touching or otherwise potentially contaminating the acetabular shell 11 itself. Lastly, when the acetabular shell 11 has been positioned at a desired location at the surgical site, the inner sheath portion 23 is removed therefrom, as shown in FIG. 7. This removal of the inner sheath portion 23 can also be accomplished quickly and easily, without touching or otherwise potentially contaminating the acetabular shell 11.

FIG. 8 is a perspective view of a second embodiment of an inner sheath portion, indicated generally at 25, in accordance with this invention. The second embodiment of the inner sheath portion 25 is similar in structure and operation to the first embodiment of the inner sheath portion 23 described above. However, the second embodiment of the inner sheath portion 25 additionally includes a plurality of apertures 25a that extends therethrough from the outer surface to the inner surface. These apertures 25a are preferably sized and shaped to correspond with the plurality of apertures 11a that extends through the acetabular shell 11. Thus, when the second embodiment of the inner sheath portion 25 is installed on the acetabular shell 11, the apertures 25a extending through the second embodiment of the inner sheath portion 25 are aligned with the apertures Ila extending through the acetabular shell 11. This arrangement permits the respective threaded fasteners (not shown) to secure the acetabular shell 11 to the hip of the patient before the second embodiment of the inner sheath portion 25 is removed from the acetabular shell 11.

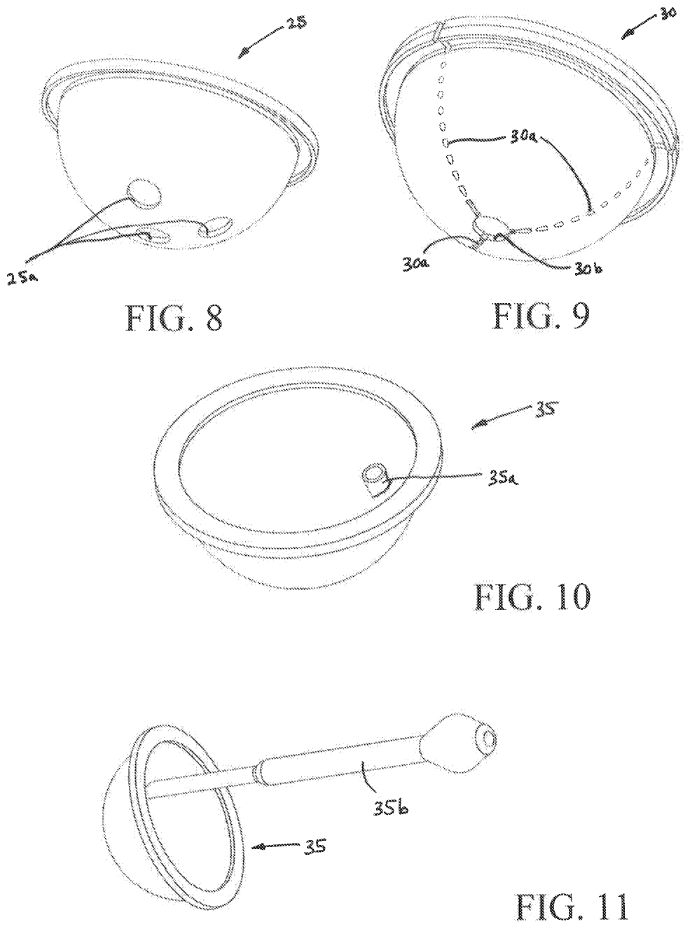

FIG. 9 is a perspective view of a third embodiment of an inner sheath portion, indicated generally at 30, in accordance with this invention. The third embodiment of the inner sheath portion 30 is similar in structure and operation to the first embodiment of the inner sheath portion 23 described above. However, the third embodiment of the inner sheath portion 30 additionally has one or more perforations 30a or similar weakened regions provided therein. The illustrated third embodiment of the inner sheath portion 30 has three of such perforations 30a provided therein that extend generally equidistantly from a central opening 30b. However, any desired number of such perforations 30a may be provided in any desired orientation. The perforations 30a are provided to facilitate removal of the third embodiment of the inner sheath portion 30 from the acetabular shell 11, as described above.

FIG. 10 is a perspective view of a fourth embodiment of an inner sheath portion, indicated generally at 35, in accordance with this invention. The fourth embodiment of the inner sheath portion 35 is similar in structure and operation to the first embodiment of the inner sheath portion 23 described above. However, the fourth embodiment of the inner sheath portion 35 additionally has a gripping structure 35a provided thereon. The illustrated gripping structure 35a is a hollow cylindrical protrusion that extends outwardly from a surface of the fourth embodiment of the inner sheath portion 35. However, the gripping structure 35a may have any desired shape or size, and further may function in any desired manner. As shown in FIG. 11, the illustrated gripping structure 35a is adapted to receive an end of a handling tool 35b therein in a releasable frictional engagement. Thus, the handling tool 35b can be used both for facilitating the securement of the acetabular shell 11 to the hip of the patient before the fourth embodiment of the inner sheath portion 35 is removed from the acetabular shell 11, and also for facilitating the removal of the fourth embodiment of the inner sheath portion 35 from the acetabular shell 11, as described above.

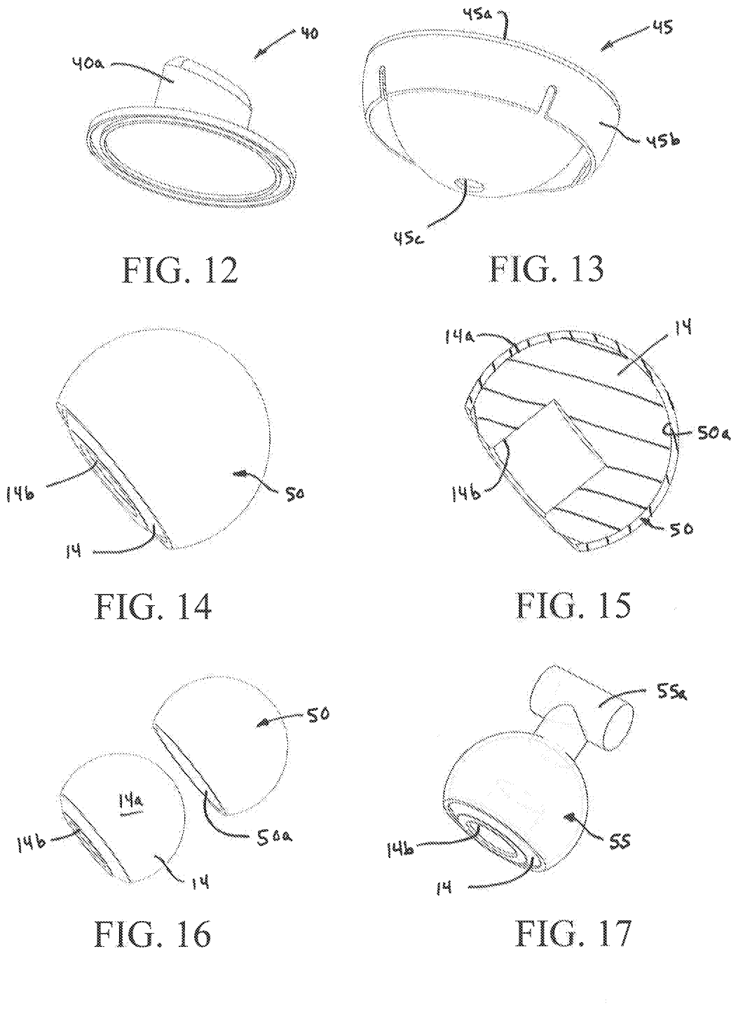

FIG. 12 is a perspective view of a fifth embodiment of an inner sheath portion, indicated generally at 40, in accordance with this invention. The fifth embodiment of the inner sheath portion 40 is similar in structure and operation to the first embodiment of the inner sheath portion 23. However, the fifth embodiment of the inner sheath portion 40 is not generally hollow and semi-spherical in shape, but rather is generally flat and circular in shape. Additionally, the fifth embodiment of the inner sheath portion 40 has a gripping structure 40a provided thereon, for the same general purpose as the gripping structure 35a described above.

FIG. 13 is a perspective view of a sixth embodiment of an inner sheath portion, indicated generally at 45, in accordance with this invention. The sixth embodiment of the inner sheath portion 45 is similar in structure and operation to the first embodiment of the inner sheath portion 23 described above. However, the L-shaped rim of the sixth embodiment of the inner sheath portion 45 includes a radially extending leg portion 45a that extends laterally across the end portion of the acetabular shell 11 and an enlarged axially extending leg portion 45b that extends across a portion of the outer surface of acetabular shell 11. The enlarged axially extending leg portion 45b protectively covers a larger portion of the outer surface of the acetabular shell 11 than does the first embodiment of the inner sheath portion 23 described above. Additionally, the sixth embodiment of the inner sheath portion 45 includes a central opening 45c.

FIGS. 14 through 16 illustrate a seventh embodiment of a protective sheath, indicated generally at 50, in accordance with this invention. As shown therein, the seventh embodiment of the sheath 50 is provided in connection with the femoral head 14 of the hip implant assembly 10, although such is not required. The femoral head 14 has an outer surface 14a that, in the illustrated embodiment, is generally spherical in shape. A generally cylindrical recess 14b is provided in the femoral head 14 that facilitates the securement of the femoral head 14 to the femoral stem 15 in a conventional manner. However, the femoral head 14 may have any desired shape and may be connected to the femoral stem 15 in any desired manner.

As best shown in FIG. 15, the seventh embodiment of the sheath 50 is generally hollow and spherical in shape, including an inner surface 50a that generally corresponds in shape with the shape of the outer surface 14a of the femoral head 14. Thus, when the seventh embodiment of the sheath 50 is installed on the femoral head 14, the inner spherical surface 50a of the sheath 50 is supported on the outer spherical surface 14a of the femoral head 14. The cooperating shapes of the outer surface 14a of the femoral head 14 and the inner surface 50a of the sheath 50 function to retain the sheath 50 on the femoral head 14, as shown in FIGS. 14 and 15.

The combined assembly of the femoral head 14 and the seventh embodiment of the sheath 50 is preferably pre-assembled in the configuration shown in FIGS. 14 and 15, sterilized as a unit, and disposed within a protective container (not shown, but which may be similar to the protective container 21 described above) for delivery to the site of the operation for implantation. Such pre-assembly and sterilization facilitate the installation of the femoral head 14 in the patient as described above, with little or no manual handling of the femoral head 14 by the surgeon or assistant. As is well known, it is very important to minimize or eliminate such manual handling of the femoral head 14 (or any other medical implant, for that matter) to reduce the likelihood of undesirable contamination.

FIG. 17 illustrates an eighth embodiment of a protective sheath, indicated generally at 55, in accordance with this invention. As shown therein, the eighth embodiment of the sheath 55 is provided in connection with the femoral head 14 of the hip implant assembly 10, although such is not required. The eighth embodiment of the sheath 55 is identical in structure and operation to the seventh embodiment of the sheath 50, except that the eighth embodiment of the sheath 55 has a handle portion 55a provided thereon to facilitate handling without manual handling by the surgeon or assistant. The handle portion 55a may be used to position the combined assembly of the femoral head 14 and the eighth embodiment of the sheath 55, and further to facilitate removal of the eighth embodiment of the sheath 55 from the femoral head 14.

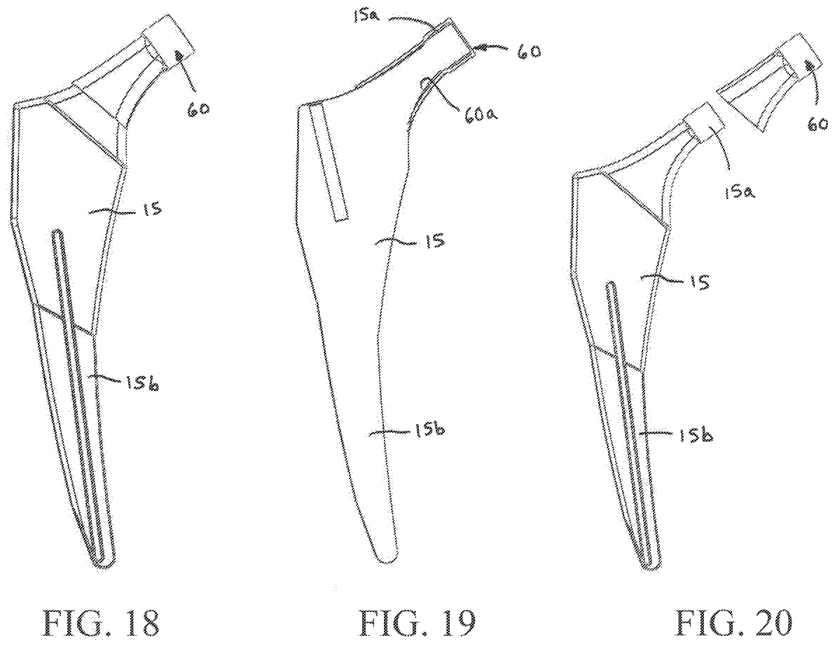

FIGS. 18 through 20 illustrate a ninth embodiment of a protective sheath, indicated generally at 60, in accordance with this invention. As shown therein, the ninth embodiment of the sheath 60 is provided in connection with the femoral stem 15 of the hip implant assembly 10, although such is not required. The femoral stem 15 includes a head portion 15a having an outer surface that, in the illustrated embodiment, is generally cylindrical in shape. The femoral stem 15 also includes a leg portion 15b. However, the femoral stem 15 may have any desired shape.

As best shown FIG. 19, the ninth embodiment of the sheath 60 is generally hollow and cylindrical in shape, including an inner surface 60a that generally corresponds in shape with the shape of the outer cylindrical surface of the head portion 15a of the femoral stem 15. Thus, when the ninth embodiment of the sheath 60 is installed on the femoral stem 15, the inner cylindrical surface 60a of the ninth embodiment of the sheath 60 is supported on the outer cylindrical surface of the head portion 15a of the femoral stem 15. The cooperating shapes of the outer cylindrical surface of the head portion 15a of the femoral stem 15 and the inner cylindrical surface 60a of the ninth embodiment of the sheath 60 function to retain the ninth embodiment of the sheath 60 on the femoral stem 15, as shown in FIGS. 18 and 19.

The combined assembly of the femoral stem 15 and the ninth embodiment of the sheath 60 is preferably pre-assembled in the configuration shown in FIGS. 18 and 19, sterilized as a unit, and disposed within a protective container (not shown, but which may be similar to the protective container 21 described above) for delivery to the site of the operation for implantation. Such pre-assembly and sterilization facilitate the installation of the femoral stem 15 in the patient as described above, with little or no manual handling of the femoral stem 15 by the surgeon or assistant. As is well known, it is very important to minimize or eliminate such manual handling of the femoral stem 15 (or any other medical implant, for that matter) to reduce the likelihood of undesirable contamination.

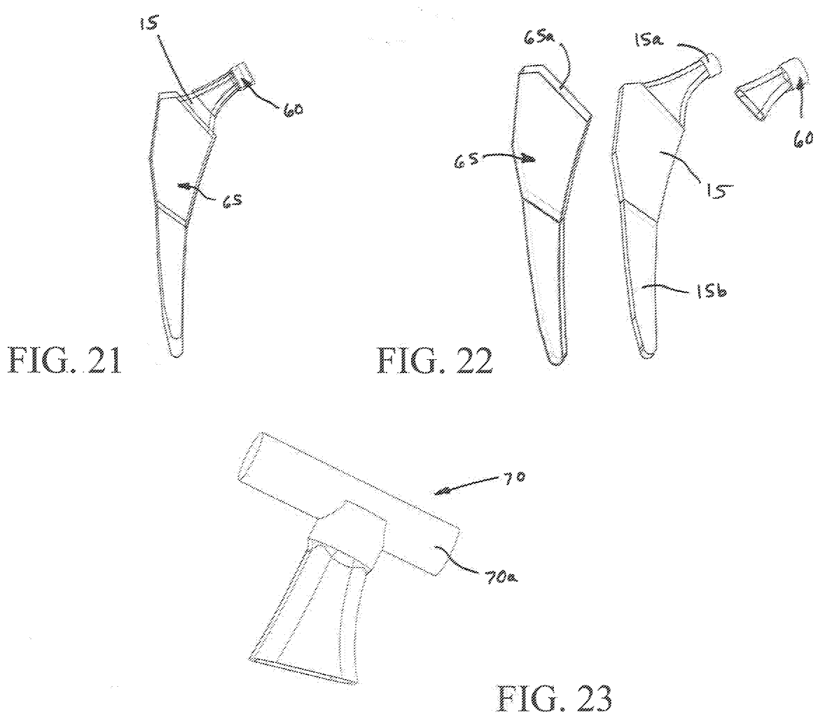

FIGS. 21 and 22 illustrate a tenth embodiment of a protective sheath, indicated generally at 65, in accordance with this invention. As shown therein, the tenth embodiment of the sheath 65 is provided in connection with the leg portion 15b of the femoral stem 15 of the hip implant assembly 10, although such is not required. The tenth embodiment of the sheath 65 includes a hollow interior 65a that is sized and shaped to receive the leg portion 15b of the femoral stem 15 therein, as shown FIG. 21. The tenth embodiment of the sheath 65 may, however, have any desired size or shape.

The combined assembly of the femoral stem 15 and the tenth embodiment of the sheath 65 is preferably pre-assembled in the configuration shown in FIG. 21, sterilized as a unit, and disposed within a protective container (not shown, but which may be similar to the protective container 21 described above) for delivery to the site of the operation for implantation. Such pre-assembly and sterilization facilitate the installation of the femoral stem 15 in the patient as described above, with little or no manual handling of the femoral stem 15 by the surgeon or assistant. As is well known, it is very important to minimize or eliminate such manual handling of the femoral stem 15 (or any other medical implant, for that matter) to reduce the likelihood of undesirable contamination.

FIG. 23 illustrates an eleventh embodiment of a protective sheath, indicated generally at 70, in accordance with this invention. As shown therein, the eleventh embodiment of the sheath 70 is provided in connection with the femoral stem 15 of the hip implant assembly 10, although such is not required. The eleventh embodiment of the sheath 70 is identical in structure and operation to the ninth embodiment of the sheath 60, except that the eleventh embodiment of the sheath 70 has a handle portion 70a provided thereon to facilitate handling without manual handling by the surgeon or assistant. The handle 70a may be used to position the combined assembly of the femoral stem 15 and the eleventh embodiment of the sheath 70, and further to facilitate removal of the eleventh embodiment of the sheath 70 from the femoral stem 15.

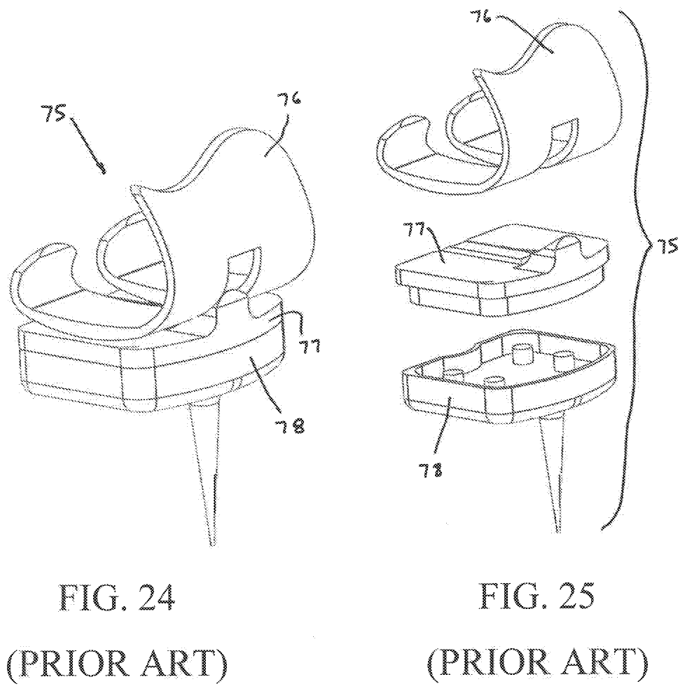

FIGS. 24 and 25 illustrate yet another medical implant, indicated generally at 75, that is conventional in the art. The illustrated conventional medical implant 75 is a knee joint assembly that includes a first knee joint component 76, a second knee joint component 77, and a third knee joint component 78, although a greater or lesser number of components may be provided. The first knee joint component 76 cooperates with the second and third knee joint components 77 and 78 so as to facilitate relative pivoting movement therebetween. It should be understood, however, that the illustrated conventional knee joint assembly 75 is intended to be representative of any individual device or combination of devices intended for installation in a human or animal body during a surgical procedure. Thus, the scope of this invention is not intended to be limited to the specific structure of the illustrated conventional knee joint assembly 75. On the contrary, as will become apparent below, this invention may be used in any desired environment and for any desired purpose.

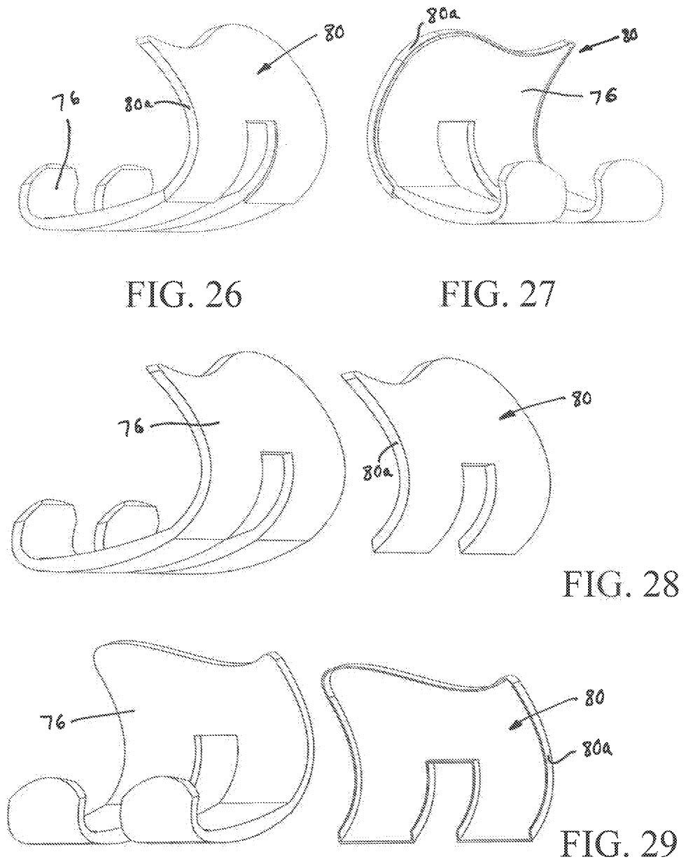

FIGS. 26 through 29 illustrate a twelfth embodiment of a protective sheath, indicated generally at 80, that can be used in connection with the first knee joint component 76 in accordance with this invention. As shown therein, the combined assembly of the first knee joint component 76 and the twelfth embodiment of the sheath 80 is preferably pre-assembled in the configuration shown in FIGS. 26 and 27, sterilized as a unit, and disposed within a protective container (not shown, but which may be similar to the protective container 21 described above) for delivery to the site of the operation for implantation. Such pre-assembly and sterilization facilitate the installation of the first knee joint component 76 in the patient as described above, with little or no manual handling of the first knee joint component 76 by the surgeon or assistant. As is well known, it is very important to minimize or eliminate such manual handling of the first knee joint component 76 (or any other medical implant, for that matter) to reduce the likelihood of undesirable contamination.

FIGS. 26 and 27 show the twelfth embodiment of the sheath 80 secured to the first knee joint component 76, while FIGS. 28 and 29 show the twelfth embodiment of the sheath 80 after removal from the first knee joint component 76. As shown therein, the twelfth embodiment of the sheath 80 includes a retaining portion 80a that facilitates the releasable securement of the twelfth embodiment of the sheath 80 to the first knee joint component 76. This releasable securement of the twelfth embodiment of the sheath 80 to the first knee joint component 76 can be accomplished by frictional engagement alone, a flexible hook-like mechanical engagement, or in any other desired manner. In the illustrated embodiment, the retaining portion 80a extends generally perpendicularly from and completely about the perimeter of the twelfth embodiment of the sheath 80, although such is not required. Rather, the retaining portion 80a may extend in any desired direction from the twelfth embodiment of the sheath 80, and further may extend about any portion or portions thereof.

FIGS. 30 through 32 illustrate a thirteenth embodiment of a protective sheath, indicated generally at 85, that can be used in connection with the second knee joint component 77 in accordance with this invention. As shown therein, the combined assembly of the second knee joint component 77 and the thirteenth embodiment of the sheath 85 is preferably pre-assembled in the configuration shown in FIGS. 30 and 32, sterilized as a unit, and disposed within a protective container (not shown, but which may be similar to the protective container 21 described above) for delivery to the site of the operation for implantation. Such pre-assembly and sterilization facilitate the installation of the second knee joint component 77 in the patient as described above, with little or no manual handling of the second knee joint component 77 by the surgeon or assistant. As is well known, it is very important to minimize or eliminate such manual handling of the second knee joint component 77 (or any other medical implant, for that matter) to reduce the likelihood of undesirable contamination.

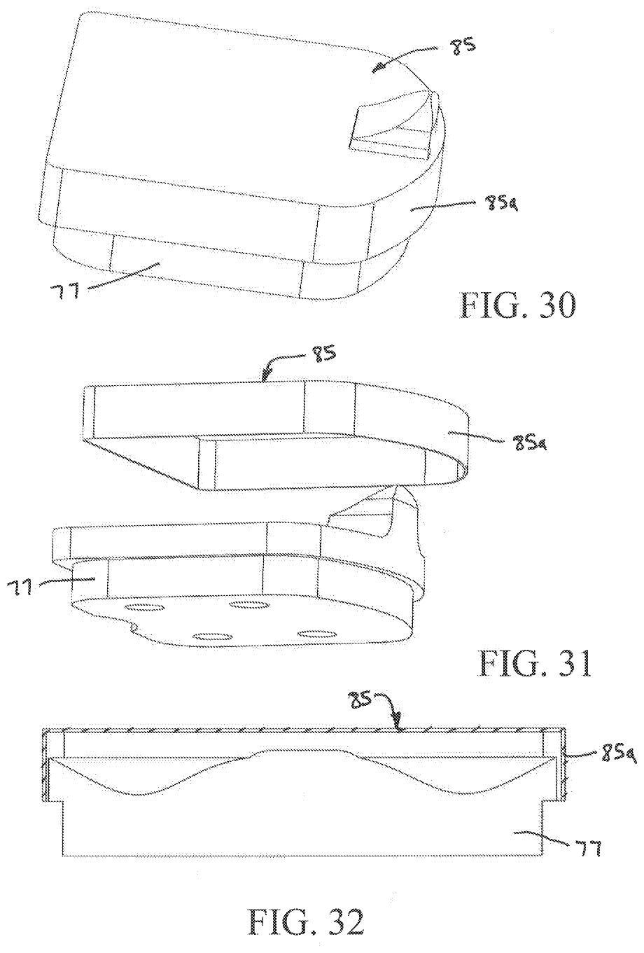

FIGS. 30 and 32 show the thirteenth embodiment of the sheath 85 secured to the second knee joint component 77, while FIG. 31 shows the thirteenth embodiment of the sheath 85 after removal from the second knee joint component 77. As shown therein, the thirteenth embodiment of the sheath 85 includes a retaining portion 85a that facilitates the releasable securement of the thirteenth embodiment of the sheath 85 to the second knee joint component 77. This releasable securement of the thirteenth embodiment of the sheath 85 to the second knee joint component 77 can be accomplished by frictional engagement alone, a flexible hook-like mechanical engagement, or in any other desired manner. In the illustrated embodiment, the retaining portion 85a extends generally perpendicularly from and completely about the perimeter of the thirteenth embodiment of the sheath 85, although such is not required. Rather, the retaining portion 85a may extend in any desired direction from the thirteenth embodiment of the sheath 85, and further may extend about any portion or portions thereof.

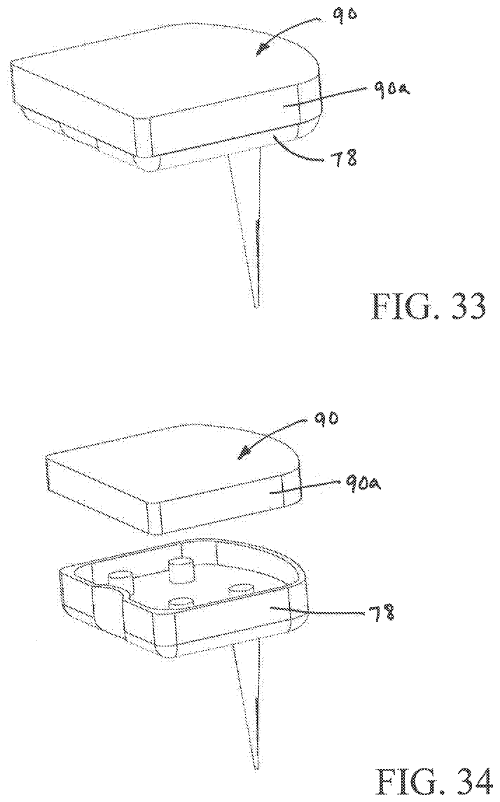

FIGS. 33 and 34 illustrate a fourteenth embodiment of a protective sheath, indicated generally at 90, that can be used in connection with the third knee joint component 78 in accordance with this invention. As shown therein, the combined assembly of the third knee joint component 78 and the fourteenth embodiment of the sheath 90 is preferably pre-assembled in the configuration shown in FIG. 33, sterilized as a unit, and disposed within a protective container (not shown, but which may be similar to the protective container 21 described above) for delivery to the site of the operation for implantation. Such pre-assembly and sterilization facilitate the installation of the third knee joint component 78 in the patient as described above, with little or no manual handling of the third knee joint component 78 by the surgeon or assistant. As is well known, it is very important to minimize or eliminate such manual handling of the third knee joint component 78 (or any other medical implant, for that matter) to reduce the likelihood of undesirable contamination.

FIG. 33 shows the fourteenth embodiment of the sheath 90 secured to the third knee joint component 78, while FIG. 34 shows the fourteenth embodiment of the sheath 90 after removal from the third knee joint component 78. As shown therein, the fourteenth embodiment of the sheath 90 includes a retaining portion 90a that facilitates the releasable securement of the fourteenth embodiment of the sheath 90 to the third knee joint component 78. This releasable securement of the fourteenth embodiment of the sheath 90 to the third knee joint component 78 can be accomplished by frictional engagement alone, a flexible hook-like mechanical engagement, or in any other desired manner. In the illustrated embodiment, the retaining portion 90a extends generally perpendicularly from and completely about the perimeter of the fourteenth embodiment of the sheath 90, although such is not required. Rather, the retaining portion 90a may extend in any desired direction from the fourteenth embodiment of the sheath 90, and further may extend about any portion or portions thereof.

Thus, in its most basic embodiment, the surface of the sheath of this invention that is in contact with the medical implant mimics the surface contour of the medical implant to a varying degree, whereas the opposite surface of the sheath of this invention that is exposed is either congruent or parallel to the formerly defined surface, or is shaped to facilitate handling and delivery, without contamination, as per the surgical technique. As used herein, the term “medical implant” is intended to refer to any component of a prosthesis that is temporarily or permanently implanted in a joint space, especially the hip replacement and knee replacement devices described and illustrated herein.

The principle and mode of operation of this invention have been explained and illustrated in its preferred embodiments. However, it must be understood that this invention may be practiced otherwise than as specifically explained and illustrated without departing from its spirit or scope.

What is claimed is:

1. A combined assembly of a medical implant and a sheath comprising:
a medical implant having a surface; and
a sheath that is releasably secured to and protectively covers the surface of the medical implant.

2. The combined assembly defined in claim 1 wherein the sheath protectively covers the entire surface of the medical implant.

3. The combined assembly defined in claim 1 wherein the sheath is frictionally retained on the medical implant.

4. The combined assembly defined in claim 3 wherein the sheath includes a generally L-shaped rim that frictionally and releasably retains the sheath on the medical implant.

5. The combined assembly defined in claim 1 wherein the sheath is mechanically retained on the implant.

6. The combined assembly defined in claim 5 wherein the sheath includes a ridge that extends into a groove provided on the medical implant to releasably and mechanically retain the sheath thereon.

7. The combined assembly defined in claim 1 wherein the medical implant includes both an inner surface and an outer surface, and wherein the sheath includes both an inner sheath portion and an outer sheath portion that are respectively releasably secured to and protectively cover the inner surface and the outer surface of the medical implant.

8. The combined assembly defined in claim 1 wherein the sheath includes one or more perforations.

9. The combined assembly defined in claim 1 wherein the sheath includes a gripping structure that cooperates a handling tool.

10. The combined assembly defined in claim 1 wherein the sheath includes a handle.

11. The combined assembly defined in claim 1 wherein the medical implant is an acetabular shell including a generally spherical outer surface, and wherein the sheath includes a generally hollow and spherical inner surface that is in engagement with the generally spherical outer surface of the acetabular shell.

12. The combined assembly defined in claim 11 wherein the acetabular shell further includes a generally semi-spherical inner surface, and wherein the sheath further includes a generally hollow and semi-spherical outer surface that is in engagement with the generally semi-spherical inner surface of the acetabular shell.

13. The combined assembly defined in claim 1 wherein the medical implant is a femoral head including a generally spherical outer surface, and wherein the sheath includes a generally hollow and spherical inner surface that is in engagement with the generally spherical outer surface of the femoral head.

14. The combined assembly defined in claim 1 wherein the medical implant is a femoral stem including a generally cylindrical head portion, and wherein the sheath includes a generally cylindrical inner surface that is in engagement with the generally cylindrical head portion of the femoral stem.

15. The combined assembly defined in claim 14 wherein the femoral stem further includes a leg portion, and wherein the sheath includes a second portion that is in engagement with the leg portion of the femoral stem.

16. The combined assembly defined in claim 1 wherein the medical implant is a component of a knee joint assembly.
